(19) United States
(12) Patent Application Publication
(10) Pub. No.: US 2015/0017256 A1
(43) Pub. Date: Jan. 15, 2015

(54) CONDENSED IRON (III) PHOSPHATE

(71) Applicant: Chemische Fabrik Budenheim KG, Budenheim (DE)

(72) Inventors: Gunnar Buehler, Nickenich (DE); Manola Stay, Mainz (DE)

(21) Appl. No.: 14/370,690

(22) PCT Filed: Jan. 2, 2013
(86) PCT No.: PCT/EP2013/050011

§ 371 Date: Jul. 3, 2014

(30) Foreign Application Priority Data
Jan. 10, 2012 (DE) 102012100128.6

(51) Int. Cl. C01B 25/37; A23L 1/304; A61K 33/42

(52) U.S. Cl. CPC C01B 25/375; A61K 33/42; A23L 1/3045; C01P 2006/82; A23V 2002/00
USPC 424/604; 423/305; 426/74; 426/648

(57) ABSTRACT

Method for producing condensed iron (III) phosphate, in which a) an aqueous solution containing Fe2+ ions is produced, in which oxidic iron (II), iron (III) or mixed iron (II, III) compounds selected from among hydroxides, oxides, oxide hydroxides, oxide hydrates, carbonates and hydroxide carbonates are introduced together with elementary iron into an aqueous medium containing phosphoric acid, wherein Fe2+ ions are dissolved and Fe3+ with elementary Fe (in a comproportionation reaction) is reacted to dissolved Fe2+, b) separating solid material from the phosphoric acid aqueous Fe2+ solution, c) adding an oxidizer to the phosphoric acid aqueous Fe2+ solution to oxidise iron (II) in the solution, d) adding polyphosphate in the form of polyphosphoric acid or salts thereof as solid material or aqueous solution after completion of the oxidation reaction to precipitate condensed iron (III) phosphate, and e) separating the precipitated condensed iron (III) phosphate solution and resulting product.

### CROSS REFERENCE TO RELATED APPLICATIONS

This application is a nationalization of International Application PCT/EP2013/050011 filed Jan. 2, 2013 and claims priority from German Application DE 102012100128.6 filed Jan. 10, 2012 both of which are incorporated by reference.

### BACKGROUND OF THE INVENTION

The invention relates to condensed iron(III) phosphate of the general formula Fe₍ₙ₊₂₎(PₙO₃ₙ₊₁)₃.xH₂O, where n≧2 and x≦9, and a process for the preparation thereof. On the basis of its properties, the condensed iron(III) phosphate is suitable inter alia for use as a foodstuffs additive for enrichment with minerals.

Iron(III) pyrophosphate (FePP) is employed inter alia as a source of iron in the field of nutrition of humans and animals and in the fertilization of plants. When employed in the diet of humans and animals, iron(III) pyrophosphate has the advantage that its influence on the sensory properties of the foodstuff is usually negligible.

Iron deficiency affects about a quarter of the world's population and can have far-reaching consequences, since iron is required for a large number of physiological functions in organisms, e.g. oxygen transport from the lungs to tissue, electron transport in cells and as a cofactor for enzymatic reactions.

The preparation of iron(III) pyrophosphate (FePP) according to the prior art is in general carried out by cation/anion exchange utilizing the low solubility product of FePP. For this, a water-soluble iron salt, usually iron sulphate, iron chloride, iron citrate or the like, is reacted with an alkali metal diphosphate, such as, for example, tetrasodium pyrophosphate (TSPP) or disodium dihydrogen pyrophosphate (sodium acid pyrophosphate, SAPP) in aqueous solution. If Fe(II) salts are used, an oxidation to Fe(III) must be carried out by addition of a suitable oxidizing agent. The initiation or completion of the precipitation of the FePP from the aqueous solution is often carried out by controlling the pH in the region of about 3.

After the precipitation the solid is conventionally separated off from the solution e.g. by filtration. If the FePP is prepared, for example, by reaction of iron chloride or iron sulphate with TSPP or SAPP, equimolar amounts of NaCl or Na₂SO₄ are formed. Such and other foreign salts are as a rule undesirable and should not remain in the end product, since they can cause trouble during later use of the product, for example by changing the colour, influencing the taste, forming lumps or undergoing undesirable chemical reactions with other formulation components. In order to remove undesirable cations and anions, the filter cake obtained must therefore be subjected to intensive washing. After the drying, FePP is obtained as a brownish to yellow-white powder, depending on the extent and nature of the contamination with foreign ions.

FePP of various levels of hydration Fe₄(P₂O₇)₃.xH₂O is obtained by the known preparation methods, whereby the solids isolated are obtained in the amorphous form and therefore cannot be characterized or identified with respect to the chemical empirical formula structurally via x-ray methods However, for characterization the contents of the main constituents Fe, P₂O₅ and H₂O (via the loss on ignition LI) can be determined. Furthermore, by means of IR spectroscopy the presence of a pyrophosphate band can be determined with the aid of a vibration of the O₃P—O—PO₃ group.

The USA FCC (Food Chemical Codex), a collection of internationally recognized monograph standards and test methods for determination of the purity and quality of foodstuffs chemicals, is based with respect to the specifications for iron(III) pyrophosphate (Ferric Pyrophosphate, FePP) on a compound of the formula Fe₄(P₂O₇)₃. 9H₂O, which is said to have, based on the ideally pure compound, the following contents (in % by weight) of the individual constituents, the contents being conventionally expressed as Fe₂O₃, P₂O₅ and H₂O (=loss on ignition LI):

Fe₄(P₂O₇)₃. 9H₂O
Fe₂O₃: 35.2%
P₂O₅: 46.9%
H₂O (LI): 17.9%

The content of iron (Fe) is 24.6%. The ratio of Fe:P₂O₅ can expediently be used as a parameter for determining the FePP content in a product which, due to its preparation, comprises not only pure FePP but also orthophosphate and/or more highly condensed phosphate forms, since this ratio is not subject to the influence of varying contents of water of hydration and surface-bonded moisture. The Fe:P₂O₅ ratio of an ideal FePP of the formula Fe₄(P₂O₇)₃. 9H₂O is accordingly 0.525.

According to the FCC, an FePP must meet the following specification:
Fe: 24.0-26.0%
LI: ≦20.0%
As: ≦3 mg/kg
Pb: ≦4 mg/kg
Hg: ≦3 mg/kg

The P₂O₅ content of an FePP which conforms to the FCC accordingly has not yet been defined. In the preparation of an FePP which conforms to the FCC, there is therefore the possibility, by significant variation in the loss on ignition (LI) in ranges significantly below the theoretical 17.9%, which can be tolerated according to the specification, of adhering to the requirement of at least 24% of Fe. The more removed the LI from the theoretical value of 17.9% of the pure FePP, the lower the probability that the compound present is an FePP with the properties envisaged according to the FCC. A low LI requires a high energy consumption during preparation of the product and results in a significantly increasing yellow-brown coloration of the product with decreasing LI, which in many cases of use is an undesirable product property.

At a minimum content of Fe of 24% according to the FCC and the maximum permitted LI of 20%, and with the corresponding Fe:P₂O₅ ratio for pure FePP of 0.525, a product which mathematically has a minimum content of 98% of FePP results.

At a maximum content of Fe of 26% according to the FCC and the maximum permitted LI of 20%, a product which mathematically comprises 42.9% of P₂O₅ results, which allows only a content of at most 81.1% of FePP. Consequently, with such a constellation compounds which do not correspond to FePP must necessarily also be present.

DE 10 2009 001 204 describes inter alia the preparation of a phosphoric acid Fe²⁺ solution, wherein oxidic compounds of iron of the most diverse nature are reacted together with elemental iron in aqueous solutions of orthophosphoric acid of various concentrations. The solution comprises exclusively dilute aqueous phosphoric acid and dissolved Fe²⁺ ions.

### BRIEF SUMMARY OF THE INVENTION

The object of the invention is the provision of novel condensed iron(III) phosphates which are distinguished inter alia by simple preparation in high purity, and a novel process for the preparation thereof and the use thereof.

The object according to the invention is achieved by a process for the preparation of condensed iron(III) phosphate of the general formula Fe₍ₙ₊₂₎(PₙO₃ₙ₊₁)₃. xH₂O, where n≧2 and x≦9, in which

a) an aqueous solution comprising Fe²⁺ ions is prepared by introducing oxidic iron(II), iron(III) or mixed iron(II,III) compounds selected from hydroxides, oxides, oxide hydroxides, oxide hydrates, carbonates and hydroxide carbonates, together with elemental iron, into an aqueous medium comprising phosphoric acid, Fe²⁺ ions being dissolved and Fe³⁺ being reacted with elemental Fe (in a comproportionation reaction) to give dissolved Fe²⁺,
b) solids present, if appropriate, are separated off from the phosphoric acid aqueous Fe²⁺ solution,
c) an oxidizing agent is added to the phosphoric acid aqueous Fe²⁺ solution in order to oxidize iron(II) in the solution, the oxidation conditions being chosen such that no iron(III) phosphates are precipitated, by keeping the temperature of the reaction solution during the addition of the oxidizing agent in the range of from 10° C. to ≦60° C. by cooling the reaction solution and/or by adjusting the rate of addition of the oxidizing agent,
d) after conclusion of the oxidation reaction polyphosphates are added to the resulting aqueous Fe³⁺ solution in the form of polyphosphoric acid or its salts as solids or as an aqueous solution, condensed iron(III) phosphate being precipitated as a solid,
e) the condensed iron(III) phosphate which has precipitated is separated off from the reaction solution.

### BRIEF DESCRIPTION OF THE SEVERAL VIEWS OF THE DRAWINGS

FIG. 1 shows an X-ray diffraction diagram of the product according to the invention according to Example 6 heat-treated at 650° C. under an air atmosphere.

FIG. 2 shows an X-ray diffraction diagram of the commercial comparison product heat-treated at 650° C. under an air atmosphere

FIG. 3 shows an infra-red spectrum of the product according to the invention according to Example 6 with typical P—O—P vibration bands at approx. 935 cm⁻¹.

### DETAILED DESCRIPTION OF THE INVENTION

In the process according to the invention, the starting substances (oxidic iron compounds, elemental iron) can be employed in powder form, preferably with particle sizes D50 in the range of from 0.01 μm to 300 μm, and mixed and reacted directly with the aqueous medium comprising phosphoric acid, preferably with dilute phosphoric acid. Alternatively, the starting substances or a portion of the starting substances can first be freshly produced via a precipitation and possibly subsequent calcining and then processed further as a filter cake. A slurry coloured or clouded (black to brown to red) by the solids content of the raw material is formed.

Where aqueous solvent is referred to here, this includes embodiments which comprise exclusively water as the liquid medium, but also those embodiments in which the liquid medium comprises water to a preferably predominant part, but can also comprise contents of water-miscible organic and/or ionic solvents or liquids. It is known that such solvent additions can have an influence on the crystal growth and therefore on the resulting morphology of the product.

In the aqueous medium comprising phosphoric acid, a redox reaction occurs between Fe³⁺ from the oxidic iron raw material and the elemental iron, soluble Fe²⁺ being formed in a comproportionation. The reaction mixture heats up by about 2 to 25° C., depending on the raw material, if the heat of reaction arising is not removed, which in principle is not necessary. After the reaction has subsided, the mixture is heated to higher temperatures, preferably below 65° C., while stirring, the solids introduced reacting more or less completely, depending on the composition and purity, to form a typically green-coloured Fe²⁺ solution. The duration of the reaction depends inter alia on the raw materials and concentrations employed.

Depending on the purity of the solids employed, a more or less pronounced clouding remains in the solution, which is caused by compounds which are insoluble under the reaction conditions. This solids content which remains can be separated off by simple filtration, sedimentation, centrifugation or other suitable means. The weights of these solids vary according to the choice of starting substances, acid concentration and reaction temperature employed in the process.

In order to remove further impurities or undesirable substances and compounds from the solution, defined precipitation reagents can advantageously be added to the solution. Thus, e.g., the calcium content in the solution can be reduced by addition of small amounts of sulphuric acid, calcium sulphate being precipitated. An additional electrolytic precipitation or deposition of undesirable metal ions out of the solution can furthermore also advantageously be carried out.

One advantage of the process according to the invention is that a homogeneous phosphoric acid aqueous iron(II) solution is prepared as an intermediate product, from which all impurities which are present as solids or can be converted into solids by precipitation additions or can be deposited electrolytically can be separated off with simple means, before the process for the preparation of the product according to the invention is continued. The product according to the invention is then not precipitated with other insoluble impurities. As a result, compared with other processes the process according to the invention allows the preparation of a product with a high purity, without particularly involved purification processes having to be subsequently carried out.

In one embodiment of the process according to the invention, the reaction of the oxidic iron compounds together with elemental iron in an aqueous medium comprising phosphoric acid is carried out at a temperature in the range of from 10° C. to 90° C., preferably in the range of from 20° C. to 75° C., particularly preferably in the range of from 25° C. to 65° C. At too low a temperature the rate of reaction is slow and possibly uneconomical. At too high a temperature a premature and undesirable precipitation of iron(III) orthophosphate may partially occur inter alia due to possible solid reactions on the solid starting substances contained in the suspension. Furthermore, the progress of side reactions such as are described below is promoted by too high a temperature.

The reaction of the oxidic iron compounds together with elemental iron in an aqueous medium comprising phosphoric acid is expediently carried out with intensive thorough mixing, preferably while stirring. All the mixers and stirrers known in the field which are suitable for such an intended use can be employed for this. Jet stream mixers, homogenizers, flow reaction cells etc. can also advantageously be used for thorough mixing and/or agitation of the reaction mixture.

In a further embodiment of the process according to the invention, the reaction of the oxidic iron compounds together with elemental iron in an aqueous medium comprising phosphoric acid is carried out for a period of time of from 1 min to 180 min, preferably from 5 min to 120 min, particularly preferably from 20 min to 90 min. The reaction of the iron compounds together with elemental iron in an aqueous medium comprising phosphoric acid can of course be interrupted at any point in time by separating off the solids from the aqueous solution, there being a loss in yield under certain circumstances if the reaction is incomplete.

In the process according to the invention the concentration of the phosphoric acid in the aqueous medium is suitably 5% to 85%, preferably 10% to 40%, particularly preferably 15% to 30%, based on the weight of the aqueous solution. Low phosphoric acid concentrations are of advantage economically, the reaction possibly proceeding very slowly at too low concentrations, which may also be undesirable from economic aspects. At high phosphoric acid concentrations, such as, for example, above 85%, formation of lumps in the oxidic iron compounds employed may occur, depending on the fineness thereof, which considerably increases the duration of the comproportionation reaction between Fe³⁺ and elemental iron described above.

Hydrogen gas forms in a side reaction between the elemental iron and the phosphoric acid, and must be removed in a controlled manner for safety reasons. This side reaction cannot be suppressed, so that a stoichiometric excess of elemental iron with respect to the amount required for the reaction of Fe³⁺ in the oxidic iron raw material should always be employed. The exact amount of this excess depends largely on the reaction conditions, such as the fineness or surface activity of the solids employed, the temperature and the acid concentration. An excess of a few per cent of the stoichiometric amount has proved to be sufficient in many cases. At temperatures above 40° C. an increase in the rate of the side reaction was observed. Above 70° C. a simultaneous precipitation of iron orthophosphate may start, so that no homogeneous Fe²⁺ solution is obtained. If the formation of lumps in the oxidic iron components already mentioned above occurs, the elemental iron largely reacts via the side reaction. The corresponding stoichiometries are therefore to be coordinated to the particular reaction conditions chosen and to the reactivity of the raw materials employed.

After the iron(II) has been dissolved out of the oxidic starting material and the iron(III) and the elemental iron have reacted by comproportionation to give iron(II) and the impurities present, if appropriate, have been removed as described above, oxidizing agent is added to the phosphoric acid Fe²⁺ solution in order to oxidize iron(II) in the solution. It is essential here that the oxidation conditions are chosen such that no iron(III) phosphates are precipitated. This is achieved by keeping the temperature of the reaction solution during the addition of the oxidizing agent in the range of from 10° C. to ≦60° C. by cooling the reaction solution and/or by adjusting the rate of addition of the oxidizing agent. Since the oxidation reaction is an exothermic reaction, the temperature can be kept in the abovementioned range by cooling the reaction mixture. At the same time or alternatively, however, too great an increase in the temperature of the reaction mixture can also be prevented by adjusting the rate of addition of the oxidizing agent, and in particular severe local overheating can be prevented by slow addition and simultaneous stirring. If the temperature increases to too high a level during the oxidation reaction, there is the risk of iron orthophosphate precipitating, which is undesirable.

The oxidation is continued until essentially the entire content of iron(II) has been oxidized to iron(III) and iron(II) can no longer be detected, or the iron(II) concentration falls below a predetermined value. During the oxidation the colour of the solution changes from green (due to the Fe²⁺ ions) to pink (due to the Fe³⁺ ions present after the oxidation). Quick tests (e.g. test sticks or test strips) known to the person skilled in the art, the accuracy of which is sufficient for the purpose of the present invention, are available for detection of iron(II) in the aqueous solution.

In a preferred embodiment of the process according to the invention, the oxidizing agent which is added in order to oxidize iron(II) in the solution is an aqueous solution of hydrogen peroxide (H₂O₂). The hydrogen peroxide solution preferably has a concentration of from 15 to 50 wt. %, particularly preferably 30 to 40 wt. %.

In alternative embodiments of the process according to the invention, the oxidizing agent which is added in order to oxidize iron(II) in the solution is a gaseous medium selected from air, pure oxygen or ozone, which is blown into the aqueous solution.

After conclusion of the oxidation reaction polyphosphates are added to the resulting aqueous Fe³⁺ solution in the form of polyphosphoric acid or its salts as solids or as an aqueous solution, condensed iron(III) phosphate precipitating according to the invention. In the context of the present invention, the term polyphosphoric acid also includes pyrophosphoric acid.

In one embodiment of the process according to the invention, the polyphosphates are added in the form of an aqueous solution of polyphosphoric acid to the aqueous Fe³⁺ solution, the polyphosphoric acid preferably having a P₂O₅ content in the range of from 74 to 80 wt. %, particularly preferably a P₂O₅ content in the range of from 76 to 78 wt. %. However, the polyphosphoric acid can also be added in undiluted form, which, however, may be associated with handling difficulties, since the undiluted polyphosphoric acid can be very viscous, depending on its P₂O₅ content, and the temperature, especially at room temperature. If the polyphosphoric acid is diluted, attention must be paid to the hydrolysis of the condensed units which starts. Hydrolysis rates as a function of temperature and pH are generally known and accessible to the person skilled in the art. The result of a complete hydrolysis of polyphosphoric acid is orthophosphoric acid.

Alternatively or in addition to the addition of polyphosphoric acid, in a further embodiment of the process according to the invention the polyphosphates are added in the form of salts of polyphosphoric acid as an aqueous solution or as a solid, preferably in the form of sodium and/or potassium salts of polyphosphoric acid, particularly preferably in the form of sodium acid pyrophosphate (Na₂H₂P₂O₇; SAPP) and/or of tetrasodium pyrophosphate (Na₄P₂O₇; TSPP).

By the addition of polyphosphates in the form of the free polyphosphoric acid or its salts, formation of the condensed phosphates of iron in the form of solids occurs, and these precipitate out of the solution and can be separated off from the liquid phase by suitable technologies. The separating off of the condensed iron(III) phosphates from the aqueous solution is preferably carried out by filtration, sedimentation, centrifugation or combinations of the abovementioned separating methods.

In a preferred embodiment of the process according to the invention, after being separated off from the reaction solution the condensed iron(III) phosphate precipitated is washed at least once or several times with water, preferably with deionized water, until a dispersion of 1 wt. % of the washed condensed iron(III) phosphate in deionized water has a conductivity of <1,000 μS/cm, preferably <500 μS/cm, particularly preferably <300 μS/cm.

Contamination, such as, for example, excess phosphate from the phosphoric acid Fe(III) solution and/or cations introduced via the polyphosphate salts, e.g. Na⁺ from TSPP or SAPP, is removed from the surface of the precipitated solids which have been separated off by the washing operation. The conductivity measured on the dispersion of the product therefore decreases with increasing removal of such contamination.

In a further preferred embodiment of the process according to the invention, the precipitated and preferably washed condensed iron(III) phosphate is dewatered or dried. This is carried out at elevated temperature and/or under reduced pressure. Alternatively, after being separated off and washed the condensed iron(III) phosphate can also advantageously be further processed in the moist form as a filter cake or dispersion having solids contents of from 1 to 90 wt. %, depending on the possible or desired efficiency of the dewatering step.

In addition to the high purity which can be achieved in the end product, the process according to the invention for the preparation of condensed iron(III) phosphate also has some ecological and economic advantages over other known processes. The mother liquor which remains after condensed iron(III) phosphate has been separated off comprises substantially no contaminating reaction products, such as, for example, sulphates or chlorides, which remain in the known processes according to the prior art in which iron sulphate or iron chloride are employed as the starting material. The mother liquor from the process according to the present invention can therefore be adjusted again to the desired concentration by addition of concentrated phosphoric acid and/or by increasing the concentrations in the solution by heating, and is thus completely recyclable into the process. The losses of P₂O₅ and Fe can thus be minimized, which makes the preparation particularly advantageous from economic and ecological aspects. Furthermore, a specific treatment of the waste water or wash water in order to remove anions (e.g. sulphate, chloride, nitrate, citrate) is dispensed with. This saves costs and avoids undesirable waste.

Products having a varying average degree of condensation can be prepared in a targeted manner by the process according to the invention. The following Table 1 describes the resulting Fe/P₂O₅ ratios for various contents of iron pyrophosphate (FePP) and iron orthophosphate (FOP) in a product. For a pure FePP having a degree of condensation of 2, a ratio of about 0.525 theoretically results. Contents of non-condensed orthophosphates (such as e.g. FOP) increase the ratio. This in turn means that products having a lower Fe/P₂O₅ ratio must have a higher average degree of condensation and comprise polyphosphates of iron having an average degree of condensation of >2.

TABLE 1
Fe/P₂O₅ ratios for various contents of FePP and FOP

| FePP (%) | FOP (%) | Fe/P₂O₅ |
|---|---|---|
| 100% | 0% | 0.5248 |
| 99% | 1% | 0.5269 |
| 98% | 2% | 0.5291 |
| 97% | 3% | 0.5312 |
| 96% | 4% | 0.5334 |
| 95% | 5% | 0.5355 |
| 94% | 6% | 0.5377 |
| 93% | 7% | 0.5399 |
| 92% | 8% | 0.5421 |
| 91% | 9% | 0.5442 |
| 90% | 10% | 0.5464 |
| 89% | 11% | 0.5487 |
| 88% | 12% | 0.5509 |
| 87% | 13% | 0.5531 |
| 86% | 14% | 0.5553 |
| 85% | 15% | 0.5576 |
| 84% | 16% | 0.5598 |
| 83% | 17% | 0.5621 |
| 82% | 18% | 0.5644 |
| 81% | 19% | 0.5666 |
| 80% | 20% | 0.5689 |
| 0% | 100% | 0.7868 |

The invention also includes a condensed iron(III) phosphate of the general formula Fe₍ₙ₊₂₎(PₙO₃ₙ₊₁)₃.xH₂O, where n≧2 and x≦9, which can be prepared or is prepared by the process described herein.

In a preferred embodiment of the invention, the condensed iron(III) phosphate according to the invention of the general formula Fe₍ₙ₊₂₎(PₙO₃ₙ₊₁)₃.xH₂O, where n≧2 and x≦9, has colour values in the Lab colour space of L≧93, a≦0.2, b≦11, preferably L≧95, a≦0, b≦7. The product according to the invention is therefore distinguished by a light, preferably white colour, which is a desirable product property in many cases of use. In contrast, many condensed iron(III) phosphates according to the prior art are yellowish to brown, and for this reason they are often unsuitable for uses where such a coloration is undesirable inter alia for aesthetic reasons, such as, for example, in the preparation of medicaments, foodstuffs etc. Furthermore, the light colour of the product according to the invention is characteristic of the high purity of the product, for example with respect to impurities due to iron hydroxide Fe(OH)₃, which has a brown colour and occurs as an impurity in some products according to the prior art due to the preparation and for this reason these are significantly darker than the products according to the invention.

In a further preferred embodiment of the invention, the condensed iron(III) phosphate according to the invention has a composition, expressed in wt. % of Fe₂O₃, wt. % of P₂O₅ and wt. % of H₂O (=loss on ignition, LI), of from 34 to 37 wt. % of Fe₂O₃, 45 to 48 wt. % of P₂O₅ and 15 to 21 wt. % of H₂O, the sum of the weight contents being 100 wt. %. Products having compositions within these ranges correspond virtually ideally to the conformity requirements of the FCC.

In a further preferred embodiment of the invention, the condensed iron(III) phosphate according to the invention has a content of Fe of ≧20 wt. % and an Fe:P₂O₅ weight ratio of from 0.400 to 0.580. A content of Fe of from 20 to 25 wt. % and an Fe:P₂O₅ weight ratio of from 0.515 to 0.540 is particularly preferred. Products having compositions within these ranges correspond virtually ideally to the conformity requirements of the FCC.

In a further preferred embodiment of the invention, the condensed iron(III) phosphate according to the invention has a chloride content (Cl⁻) and/or a nitrate content (NO₃⁻) and/or a sulphate content (SO₄²⁻) and/or a sodium content (Na⁺) of <1,000 ppm, preferably <500 ppm, particularly preferably <100 ppm. The condensed iron(III) phosphate according to the invention can be prepared by the process according to the invention in a very high purity with respect to the abovementioned foreign ions. Too high contents of the foreign ions mentioned can cause trouble during later use of the product, for example by changing the colour, influencing the taste, forming lumps or undergoing undesirable chemical reactions with other formulation components.

The condensed iron(III) phosphates according to the invention of the general formula Fe₍ₙ₊₂₎(PₙO₃ₙ₊₁)₃.xH₂O, where n≧2 and x≦9, are preferably amorphous or x-ray amorphous, i.e. they deliver no reflexes in the x-ray diffraction diffractogram.

The condensed iron(III) phosphates according to the invention can be converted into a crystalline phase by heat treatment. In order to investigate this, products according to the invention according to Example 6 (see below) and commercial comparison products were heat-treated at temperatures of 150° C., 250° C., 350° C., 450° C., 550° C. and 650° C. for 2 hours under an air atmosphere and then cooled to room temperature, also under an air atmosphere. At the heat treatment temperatures of 550° C. and 650° C. the originally pulverulent samples solidified, so that for the further investigations they had to be comminuted with a mortar.

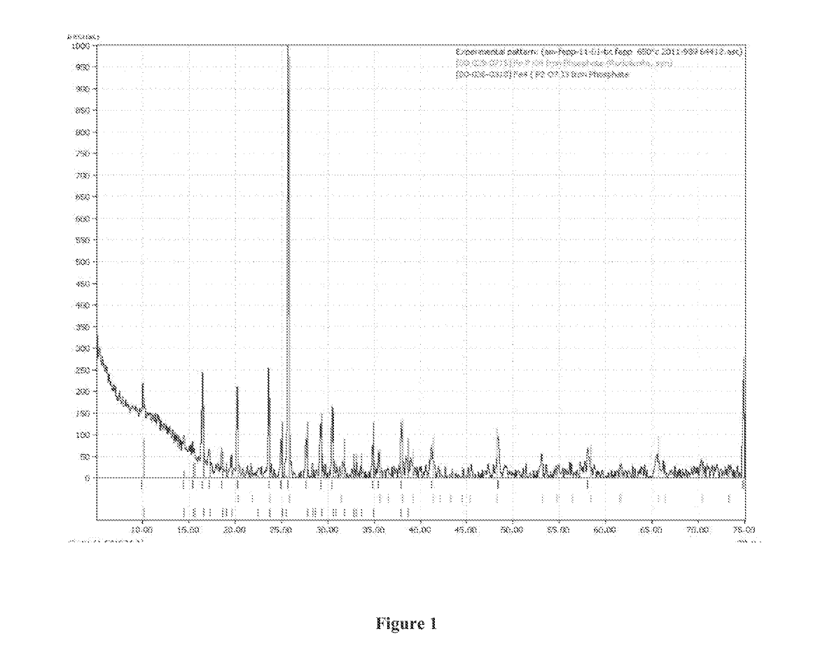

It was found that the product according to the invention first crystallized as anhydrous FePP at a temperature above 550° C. In the x-ray diffraction diagram, reflexes of anhydrous iron orthophosphate (FePO₄) were also detectable, which was attributed to a partial decomposition of the starting compound to form volatile decomposition products. The x-ray diffraction diagram obtained could be characterized with the aid of the PDF cards 036-0318 for anhydrous Fe₄(P₂O₇)₃ and 029-0715 for FePO₄. The x-ray diffraction diagram of the product according to the invention heat-treated at 650° C. is reproduced in FIG. 1.

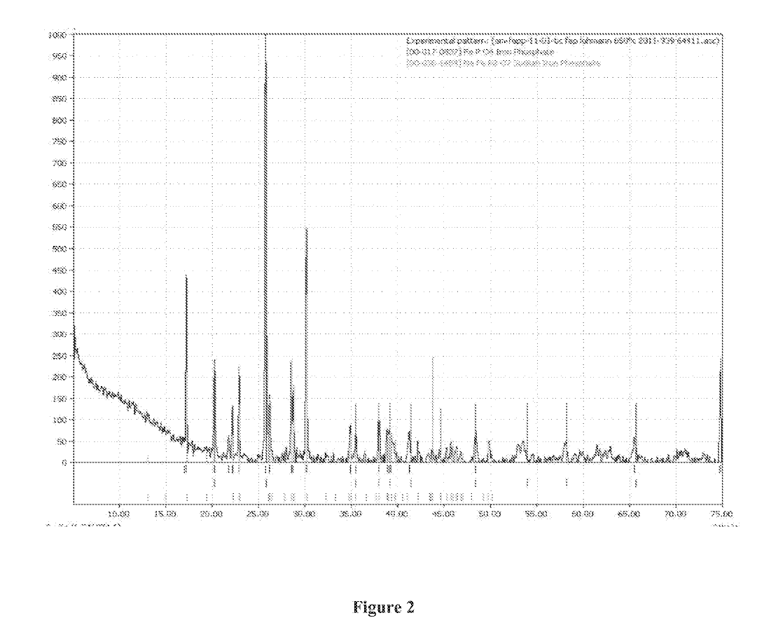

In the case of the commercial comparison product, crystallization already started at a temperature of 550° C. However, the x-ray diffraction diagram shows only reflexes of anhydrous NaFeP₂O₇, from which a high sodium content in the comparison material can be concluded. As in the case of the product according to the invention, the reflexes of anhydrous iron orthophosphate (FePO₄) also manifest themselves due to a partial decomposition of the starting compound. The x-ray diffraction diagram obtained could be characterized with the aid of the PDF cards 036-1454 for NaFe(P₂O₇) and 017-0837 for FePO₄. The x-ray diffraction diagram of the commercial comparison product heat-treated at 650° C. is reproduced in FIG. 2.

In a preferred embodiment of the invention, the condensed iron(III) phosphate according to the invention of the general formula Fe₍ₙ₊₂₎(PₙO₃ₙ₊₁)₃.xH₂O, where n≧2 and x≦9, therefore has, after heat treatment under an air atmosphere at a temperature of 650° C. for a period of time of 2 hours, peaks in the powder x-ray diffraction spectrum at 10.02±0.2, 16.44±0.2, 23.58±0.2, 24.88±0.2, 27.56±0.2, 29.14±0.2, 30.42±0.2 and 34.76±0.2 degree two-theta, based on CuKα radiation. These are typical peaks for Fe₄(P₂O₇)₃ according to PDF card 036-0318. Furthermore, the condensed iron(III) phosphate according to the invention preferably has, after the heat treatment described above, further peaks in the powder x-ray diffraction spectrum at 20.16±0.2, 25.66±0.2, 37.86±0.2, 41.14±0.2, 47.30±0.2, 48.28±0.2, 52.00±0.2 and 57.10±0.2 degree two-theta. These are typical peaks for FePO₄ according to PDF card 029-0715.

The invention furthermore includes the use of condensed iron(III) phosphate according to the invention of the general formula Fe(n+2)(PₙO₃ₙ₊₁)₃xH₂O, where n≧2 and x≦9, as a foodstuffs additive, foodstuffs supplement, additive for enriching foodstuffs for humans and animals with iron, pharmaceutical or pharmaceutically active constituent in pharmaceutical formulations for human medicine and veterinary medicine purposes, source of iron in chemical or biological systems and/or for the production of lithiumated (Li-containing) cathode material for Li ion accumulators. On the basis of its high purity, the product according to the invention is significantly superior to many known products according to the prior art for the abovementioned uses.

The invention furthermore includes a process for the preparation of a stabilized Fe³⁺ solution, in which

a) an aqueous solution comprising Fe²⁺ ions is prepared by introducing oxidic iron(II), iron(III) or mixed iron(II,III) compounds selected from hydroxides, oxides, oxide hydroxides, oxide hydrates, carbonates and hydroxide carbonates, together with elemental iron, into an aqueous medium comprising phosphoric acid, Fe²⁺ ions being dissolved and Fe³⁺ being reacted with elemental Fe (in a comproportionation reaction) to give dissolved Fe²⁺,
b) solids present, if appropriate, are separated off from the phosphoric acid aqueous Fe²⁺ solution,
c) an oxidizing agent is added to the phosphoric acid aqueous Fe²⁺ solution in order to oxidize iron(II) in the solution, the oxidation conditions being chosen such that no iron(III) phosphates are precipitated, by keeping the temperature of the reaction solution during the addition of the oxidizing agent in the range of from 10° C. to ≦60° C. by cooling the reaction solution and/or by adjusting the rate of addition of the oxidizing agent.

The process can advantageously be supplemented by a procedure in which, before or after the solids present, if appropriate, have been separated off from the phosphoric acid aqueous Fe²⁺ solution in stage b), precipitation reagents are added to the phosphoric acid aqueous solution obtained, in order to precipitate solids out of the solution, and/or metals dissolved in the phosphoric acid aqueous solution are deposited electrolytically out of the solution.

The invention furthermore includes a stabilized Fe³⁺ solution which can be prepared or is prepared by the abovementioned process. The stabilized Fe³⁺ solution according to the invention is suitable as a precursor for the preparation of the most diverse products, for example for the preparation of iron orthophosphate. It is distinguished by a high purity, so that an expensive purification before a further processing or of the products prepared therefrom as a rule is not necessary. The solution is moreover stable for a relatively long period of time, although iron(III) phosphate does not have a particularly good solubility in water. The high stability of the solution is attributed to a complexing of the Fe³⁺ ions in the solution.

The following examples serve to illustrate the invention

Preparation of the Fe³⁺ solution

700 g of 75% strength H₃PO₄ and 1,580 ml of deionized water were initially introduced into a glass beaker. 60 g of Fe powder and 75 g of Fe₂O₃ were added, while stirring, a temperature of about 30° C. being established. The mixture was then stirred at 60° C. for 2 h and thereafter the resulting green solution was freed from suspended substances present, if appropriate, by filtration. The Fe²⁺ ions present in the cooled solution were oxidized by slow addition of 220 ml of 35% strength H₂O₂ solution, while stirring, to give an Fe³⁺ solution, the H₂O₂ solution being metered in at a rate such that the temperature did not rise above 50° C.

The Fe solution prepared in this way was employed in the following examples. The precipitation of the iron(III) polyphosphate was carried out in Examples 1 to 3 with TSPP (Example 1), SAPP (Example 2) or a combination of TSPP and SAPP (Example 3). Various equivalents of a polyphosphoric acid with 76-78% of P₂O₅ were employed in Examples 4 to 7.

### Example 1

### (TSPP)

87 g of Fe³⁺ solution (0.056 mol of Fe) were initially introduced into the reaction vessel. 14.89 g of TSPP (0.056 mol) were dissolved in 200 ml of completely deionized water (DI water) and the solution was added to the stirred Fe³⁺ solution. A white precipitate precipitated. The pH of the solution was 2.3. The precipitate was filtered off, washed with DI water and dried. The experiment was repeated 2 times.

| Analysis | Repetition 1: | Repetition 2: |
|---|---|---|
| P₂O₅ (%): | 48.7 | 52.1 |
| Fe (%): | 22.3 | 24.3 |
| LI (%): | 19.4 | 12.3 |
| Fe/P₂O₅: | 0.457 | 0.465 |
| Yield: | 10.7 g | 11.3 g |

### Example 2

### (SAPP)

87 g of Fe³⁺ solution (0.056 mol of Fe) were initially introduced into the reaction vessel. 12.46 g of SAPP (0.056 mol) were dissolved in 120 ml of DI water and the solution was added to the stirred Fe³⁺ solution. A white precipitate precipitated. The pH of the solution was adjusted to 2.5 with 50% strength NaOH. The precipitate was filtered off, washed with DI water and dried. The experiment was repeated without addition of NaOH.

| | Repetition 1 | Repetition 2 |
|---|---|---|
| Analysis | (with NaOH): | (without NaOH): |
| P₂O₅ (%): | 51.1 | 52.2 |
| Fe (%): | 21.9 | 23.8 |
| LI (%): | 15.8 | 14.1 |
| Fe/P₂O₅: | 0.429 | 0.455 |
| Yield: | 14.5 g | 14.4 g |

### Example 3

### (TSPP+SAPP)

307.6 g of a 3.59% strength Fe²⁺ solution (0.198 mol of Fe) were initially introduced into the reaction vessel and oxidized completely with a 35% strength H₂O₂ solution as described above. 21.9 g of SAPP (0.099 mol) and 26.3 g of TSPP (0.099 mol) were dissolved in 380 ml of DI water and the solution was added to the stirred Fe³⁺ solution. A white precipitate precipitated, and was filtered off, washed with DI water and dried overnight at 55° C. to give a white powder.

| Analysis | |
|---|---|
| P₂O₅ (%): | 53.0 |
| Fe (%): | 21.8 |
| LI (%): | 15.5 |
| Fe/P₂O₅: | 0.411 |
| Yield: | 46.7 g |

### Example 4

(1 equivalent of pyrophosphoric acid)

10.0 g of H₄P₂O₇ (0.056 mol) were dissolved in 50 ml of DI water. 56.8 g of the 5.51% strength Fe³⁺ solution (0.056 mol of Fe) were added to the stirred pyrophosphoric acid solution. A white precipitate precipitated. The pH of the solution was 0. The precipitate was filtered off, washed with DI water and dried.

| Analysis | |
|---|---|
| P₂O₅ (%): | 46.0 |
| Fe (%): | 23.2 |
| LI (%): | 20.9 |
| Fe/P₂O₅: | 0.504 |
| Yield: | 8.7 g |

### Example 5

(0.8 equivalent of pyrophosphoric acid)

8.0 g of H₄P₂O₇ (0.045 mol) were dissolved in 50 ml of DI water. 56.8 g of the 5.51% strength Fe³⁺ solution (0.056 mol of Fe) were added to the stirred pyrophosphoric acid solution. A white precipitate precipitated. The pH of the solution was 0. The precipitate was filtered off, washed with DI water and dried.

| Analysis | |
|---|---|
| P₂O₅ (%): | 46.3 |
| Fe (%): | 23.7 |
| LI (%): | 20.6 |
| Fe/P₂O₅: | 0.512 |
| Yield: | 11.0 g |

### Example 6

(0.6 equivalent of pyrophosphoric acid)

56.8 g of the 5.51% strength Fe³⁺ solution (0.056 mol of Fe) were initially introduced into the reaction vessel. 12.5 g of H₄P₂O₇ (0.034 mol) were added to the stirred Fe³⁺ solution. 50 ml of water were then added. A white precipitate precipitated. The pH of the solution was 0. The precipitate was filtered off, washed with DI water and dried.

| Analysis | |
|---|---|
| P₂O₅ (%): | 45.8 |
| Fe (%): | 24.0 |
| LI (%): | 19.9 |
| Fe/P₂O₅: | 0.524 |
| Yield: | 8.5 g |

### Example 6

was repeated nine times (repetitions 6.0 to 6.8). The results of the Lab value evaluation are described below.

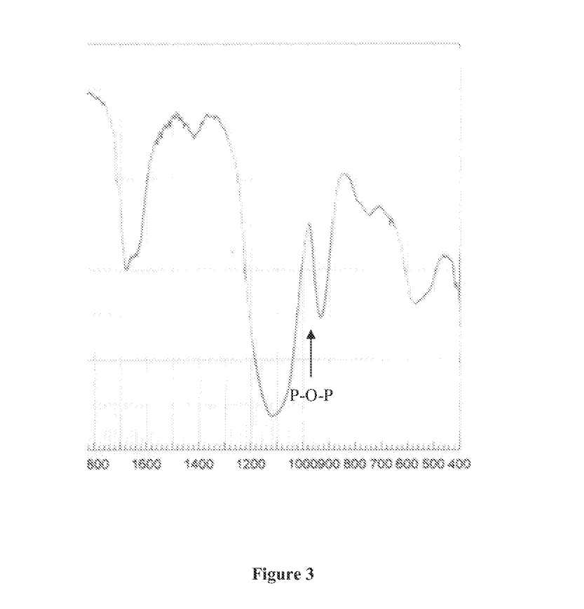

FIG. 3 shows an infra-red spectrum of the product according to the invention according to Example 6 (repetition 6.0) with typical P—O—P vibration bands at approx. 935 cm⁻¹.

### Example 7

(0.5 equivalent of pyrophosphoric acid)

100.0 g of the 5.51% strength Fe³⁺ solution (0.1 mol of Fe) were initially introduced into the reaction vessel. 10.4 g of H₄P₂O₇ (0.034 mol) were dissolved in 50 ml of water and the solution was added to the stirred Fe³⁺ solution. A white precipitate precipitated. The pH of the solution was 0. A further 50 ml of water were added. Further white precipitate precipitated. The precipitate was filtered off, washed with DI water and dried.

| Analysis | |
|---|---|
| P₂O₅ (%): | 45.7 |
| Fe (%): | 24.2 |
| LI (%): | 19.7 |
| Fe/P₂O₅: | 0.530 |
| Yield: | 13.6 g |

The embodiment examples show that iron(III) polyphosphates having Fe/P₂O₅ ratios of between 0.411 (high degree of condensation) and 0.530 can be prepared using the process according to the invention, a ratio of 0.525 corresponding exactly to a pure FePP.

Above all iron(III) polyphosphates which have been prepared using polyphosphoric acid show only low contamination (traces) with alkali metals and anions, such as sulphate, chloride, nitrate or citrate, as a result of the process.

The following Table 2 shows comparative analyses of iron(III) phosphates having a degree of condensation of 2. The commercial comparison samples were obtained from 3 different sources. The samples according to the invention are repetitions 6.0, 6.5 and 6.8 according to Example 6. The results show that none of the commercial products even only approximately reaches the Fe:P₂O₅ ratio of 0.525 of pure FePP and therefore the degree of condensation of about 2 also cannot be present in these products. Furthermore, the commercial comparison samples have considerable amounts of contamination by sulphate and sodium, whereas the products according to the invention have a very high purity.

TABLE 2
Comparative analyses of iron polyphosphates

| | | | | Samples according to the | | |
|---|---|---|---|---|---|---|
| | Commercial comparison | | | invention | | |
| | samples | | | according to Example 6 | | |
| | 1 | 2 | 3 | 6.0 | 6.5 | 6.8 |
| Fe [%] | 25 | 24 | 21.9 | 24.1 | 24.7 | 24.4 |
| P₂O₅ [%] | 35.3 | 41.1 | 45 | 45.9 | 46.9 | 45.9 |
| Fe:P₂O₅ | 0.708 | 0.584 | 0.487 | 0.525 | 0.527 | 0.532 |
| As [ppm] | <3 | <3 | <3 | <3 | <3 | <3 |
| Pb [ppm] | <4 | <4 | <4 | <4 | <4 | <4 |
| Cd [ppm] | <1 | <1 | <1 | <1 | <1 | <1 |
| Hg [ppm] | <1 | <1 | <1 | <1 | <1 | <1 |
| SO₄²⁻ [%] | 7.9 | 2.4 | 0.2 | <0.002 | <0.003 | <0.002 |
| Na⁺ [%] | 2.7 | 1.1 | 5.5 | 0.0055 | 0.0055 | 0.0060 |

In a suitable reaction procedure, products having an Fe/P₂O₅ ratio of <0.411 can also be obtained.

Determination of Lab values (L*a*b*)

The L*a*b* colour space is a measurement space containing all perceivable colours. The colour space is constructed on the basis of the contrasting colour theory. One of the most important properties of the L*a*b* colour model is that it is independent of equipment, that is to say the colours are defined independently of the nature of their generation and reproduction technique. The corresponding German standard is DIN 6174: “Colorimetric evaluation of colour coordinates and colour differences according to the approximately uniform CIELAB colour space”. The L*a*b* colour space is described by a three-dimensional coordinate system. The a* axis describes the green or red content of a colour, negative values representing green and positive values red. The b* axis describes the blue or yellow content of a colour, negative values representing blue and positive values yellow. The scales of the a* axis and the b* axis comprise a numerical range of from −150 to +100 and −100 to +150 respectively, regardless of the fact that for some values there is no perceivable equivalent. The L* axis describes the lightness (luminance) of the colour with values of from 0 to 100 [source: Wikipedia].

The following Table 3 shows the L*a*b* values of an experimental series (repetitions 6.0 to 6.8 according to Example 6) compared with a commercial reference material according to the prior art.

TABLE 3
Lab values

| | 6.0 | 6.1 | 6.2 | 6.3 | 6.4 | 6.5 | 6.6 | 6.7 | 6.8 | Reference |
|---|---|---|---|---|---|---|---|---|---|---|
| L | 95.7 | 96.1 | 96.2 | 96.4 | 96.4 | 96.4 | 96.1 | 96.3 | 96.3 | 93.9 |
| a | −0.81 | −0.77 | −0.82 | −0.8 | −0.82 | −0.83 | −0.89 | −0.88 | −0.88 | 0.35 |
| b | 5.35 | 5.12 | 5.69 | 5.56 | 5.13 | 5.07 | 5.41 | 5.01 | 5.81 | 11.18 |

The higher L values (luminance) of repetitions 6.0 to 6.8 show a significantly higher lightness of the products according to the invention, and the lower b values show a considerably lower yellow shift. For the red-green content (a value), no significant influence is found. Generally, it was found that the products prepared according to the invention all have a virtually pure white colour, in contrast to FePP according to the prior art.

1. A process for the preparation of condensed iron(III) phosphate of the general formula Fe₍ₙ₊₂₎(PₙO₃ₙ₊₁)₃.xH₂O, where n≧2 and x≦9, comprising:
a) preparing an aqueous solution comprising Fe²⁺ ions by introducing oxidic iron(II), iron(III) or mixed iron(II,III) compounds selected from the group consisting of hydroxides, oxides, oxide hydroxides, oxide hydrates, carbonates and hydroxide carbonates, together with elemental iron, into an aqueous medium comprising phosphoric acid, Fe²⁺ ions being dissolved and Fe³⁺ being reacted with elemental Fe in a comproportionation reaction to give dissolved Fe²⁺,
b) separating solids from the phosphoric acid aqueous Fe²⁺ solution when present,
c) adding an oxidizing agent to the phosphoric acid aqueous Fe²⁺ solution in order to oxidize iron(II) in the solution, the oxidation conditions being chosen such that no iron(III) phosphates are precipitated, by at least one of keeping the temperature of the reaction solution during the addition of the oxidizing agent in the range of from 10° C. to ≦60° C. by cooling the reaction solution and adjusting the rate of addition of the oxidizing agent,
d) after conclusion of the oxidation reaction, adding polyphosphates to the resulting aqueous Fe³⁺ solution in the form of polyphosphoric acid, solid salts thereof or as an aqueous solution, to precipitate condensed iron(III) phosphate as a solid,
e) separating condensed iron(III) phosphate which has precipitated from the reaction solution.

2. A process according to claim 1, wherein
f) after being separated from the reaction solution the precipitated condensed iron(III) phosphate is washed at least once with water, until a dispersion of 1 wt. % of the washed condensed iron(III) phosphate in deionized water has a conductivity of <1,000 μS/cm, preferably <500 μS/cm, particularly preferably <300 μS/cm.

3. A process according to claim 2 wherein the precipitated washed condensed iron(III) phosphate is dewatered.

4. A process according to claim 1 wherein the oxidizing agent added to the phosphoric acid aqueous Fe²⁺ solution in order to oxidize iron(II) in the solution is an aqueous solution of hydrogen peroxide (H₂O₂), preferably having a concentration of from 15 to 50 wt. % of H₂O₂, particularly preferably 30 to 40 wt. % of H₂O₂.

5. A process according to claim 1 further including at least one of the steps of adding precipitation reagents to the phosphoric acid aqueous solution obtained in stage a) or stage b), in order to precipitate solids out of the solution, and electrolytically depositing metals dissolved in the phosphoric acid aqueous solution out of the solution.

6. A process according to claim 1 wherein the reaction of the oxidic iron compounds together with elemental iron in an aqueous medium comprising phosphoric acid in stage a) is carried out under at least one of the following conditions:
a temperature in the range of from 10° C. to 90° C., preferably in the range of from 20° C. to 75° C., particularly preferably in the range of from 25° C. to 65° C.,
with intensive thorough mixing and
for a period of from 1 min to 180 min, preferably from 5 min to 120 min, particularly preferably from 20 min to 90 min.

7. A process according to claim 1 wherein the concentration of the phosphoric acid in the aqueous medium comprising phosphoric acid in stage a) is 5% to 85%, preferably 10% to 40%—particularly preferably 15% to 30%, based on the weight of the aqueous solution.

8. A process according claim 1 wherein the addition of the polyphosphates to the aqueous Fe³⁺ solution obtained after conclusion of oxidation in step c) is carried out by at least one of:
i) in the form of an aqueous solution of polyphosphoric acid, the polyphosphoric acid preferably having a P₂O₅ content in the range of from 74 to 80 wt. %, particularly preferably a P₂O₅ content in the range of from 76 to 78 wt. %, and
ii) in the form of salts of polyphosphoric acid as an aqueous solution or as a solid, preferably—in the form of sodium and/or potassium salts of polyphosphoric acid, particularly preferably in the form of sodium acid pyrophosphate (Na₂H₂P₂O₇; SAPP) and/or of tetrasodium pyrophosphate (Na₄P₂O₇; TSPP).

9. Condensed iron(III) phosphate of the general formula Fe₍ₙ₊₂₎(PₙO₃ₙ₊₁)₃.xH₂O, where n≧2 and x≦9, prepared according to the process of claim 1.

10. Condensed iron(III) phosphate of the general formula Fe₍ₙ₊₂₎(PₙO₃ₙ₊₁)₃.xH₂O, where n≧2 and x≦9, according to claim 9, wherein it has colour values in the Lab colour space of L≧93, a≦0.2, b≦11, preferably L≧95, a≦0, b≦7.

11. Condensed iron(III) phosphate of the general formula Fe₍ₙ₊₂₎(PₙO₃ₙ₊₁)₃.xH₂O, where n≧2 and x≦9, according to claim 9, wherein the iron(III) phosphate has at least one of the following properties:
the iron(III) phosphate has a composition, expressed in wt. % of Fe₂O₃, wt. % of P₂O₅ and wt. % of H₂O, the wt. % of H₂O corresponding to loss on ignition, of from 34 to 37 wt. % of Fe₂O₃, 45 to 48 wt. % of P₂O₅ and 15 to 21 wt. % of H₂O, the sum of the weight contents being 100 wt. %, and/or
the iron(III) phosphate has a content of Fe of ≧20 wt. % and an Fe:P₂O₅ weight ratio of from 0.400 to 0.580, preferably a content of Fe of from 20 to 25 wt. % and an Fe:P₂O₅ weight ratio of from 0.515 to 0.540.

12. Condensed iron(III) phosphate of the general formula Fe₍ₙ₊₂₎(PₙO₃ₙ₊₁)₃.xH₂O, where n≧2 and x≦9, according to claim 9 wherein the content of at least one of chloride (Cl⁻), nitrate (NO₃⁻), sulphate (SO₄²⁻) and sodium (Na⁺) of the iron(III) phosphate is <1,000 ppm, preferably <500 ppm, particularly preferably <100 ppm.

13. Condensed iron(III) phosphate of the general formula Fe₍ₙ₊₂₎(PₙO₃ₙ₊₁)₃.xH₂O, where n≧2 and x≦9, according to claim 9 wherein the iron(III) phosphate has at least one of the following properties:
the iron(III) phosphate is amorphous or x-ray amorphous,
after heat treatment under an air atmosphere at a temperature of 650° C. for a period of time of 2 hours, the iron(III) phosphate has peaks in the powder x-ray diffraction spectrum at 10.02±0.2, 16.44±0.2, 23.58±0.2, 24.88±0.2, 27.56±0.2, 29.14±0.2, 30.42±0.2 and 34.76±0.2 degree two-theta, based on CuKα radiation, and preferably further peaks at 20.16±0.2, 25.66±0.2, 37.86±0.2, 41.14±0.2, 47.30±0.2, 48.28±0.2, 52.00±0.2 and 57.10±0.2 degree two-theta.

14. A method for the preparation of at least one product selected from the group consisting of a foodstuffs additive, a foodstuffs supplement, an additive for enriching foodstuffs for humans and animals with iron, a pharmaceutical or pharmaceutically active constituent in pharmaceutical formulations for human medicine and veterinary medicine purposes, a source of iron in chemical or biological systems and lithiumated (Li-containing) cathode material for Li ion accumulators.

15. A process Process for the preparation of a stabilized Fe³⁺ solution, in which
a) an aqueous solution comprising Fe²⁺ ions is prepared by introducing oxidic iron(II), iron(III) or mixed iron(II,III) compounds selected from hydroxides, oxides, oxide hydroxides, oxide hydrates, carbonates and hydroxide carbonates, together with elemental iron, into an aqueous medium comprising phosphoric acid, Fe²⁺ ions being dissolved and Fe³⁺ being reacted with elemental Fe (in a comproportionation reaction) to give dissolved Fe²⁺,
b) separating solids from the phosphoric acid aqueous Fe²⁺ solution when present,
c) adding an oxidizing agent to the phosphoric acid aqueous Fe²⁺ solution in order to oxidize iron(II) in the solution, the oxidation conditions being chosen such that no iron(III) phosphates are precipitated, by at least one of keeping the temperature of the reaction solution during the addition of the oxidizing agent in the range of from 10° C. to ≦60° C. by cooling the reaction solution and by adjusting the rate of addition of the oxidizing agent.

16. A process according to claim 15, further including at least one of the steps of adding precipitation reagents to the phosphoric acid aqueous solution obtained in stage a) or stage b) in order to precipitate solids out of the solution, and electrolytically depositing metals dissolved in the phosphoric acid aqueous solution out of the solution.

17. Stabilized Fe³⁺ solution prepared according to claim 15.
